(19) United States
(12) Patent Application Publication
(10) Pub. No.: US 2002/0107578 A1
(43) Pub. Date: Aug. 8, 2002

(54) Metallic osteosynthesis aid

(76) Inventors: Andreas Werner Speitling, Kiel (DE); Geert Von Oldenburg, Kiel (DE)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/061,098

(22) Filed: Feb. 1, 2002

(30) Foreign Application Priority Data
Feb. 3, 2001 (DE) 201 01 917.5

(51) Int. Cl. A61F 2/28

(52) U.S. Cl. 623/23.6; 623/23.53

(57) ABSTRACT

A static trauma or fracture fixation device is made from a metal such as steel, cobalt-chrome alloy or titanium alloy. When in use, the implant has at least one portion which is either in contact with bone tissue or with metal. Although such a metal may be considered a static implant, it undergoes dynamic loads which cause micro-motion. In order to reduce wear, corrosion and modify the frictional characteristics of the surface, coatings may be applied. The coating may be a diamond-like carbon coating of the metal, carbon and hydrogen type. This coating may be applied by physical vapor deposition and/or chemical vapor deposition. Other coating processes may include nitrogen or oxygen diffusion processes or carbon diffusion processes. Anodization may also be used as a coating to modify the wear and corrosion resistance of the base metal.

### BACKGROUND OF THE INVENTION

The invention relates to an osteosynthesis aid used in trauma applications made from a steel, cobalt and/or titanium alloys. More particularly, they relate to metallic orthopedic implants used in joint replacement and in trauma treatment.

Osteosynthesis aids in the form of prosthetic implants are well known in the fields of traumatology and endoprosthesis, as well as being used with external fixators. The implants used are exposed to corrosion, friction and wear in the patient's body. In the past, trauma devices such as intramedullary nails, hip fracture fixation devices, bone plates and external fracture fixation devices, while being made of corrosion resistant material, were not treated with coatings to enhance wear because such devices were considered to be static device (non-moving) rather than dynamic devices. It has been discovered that during use such devices undergo dynamic movements which are small and amplitude and are referred to as micro-motion. The small movements can cause surface wear and fretting between individual components of the trauma devices and between the devices and bone. Metal ion release from the metal substrate occurs which can cause adverse tissue reactions. In the past this was believed not to be a problem with static trauma devices. Corrosion and wear damage occurs especially between individual implant components, which can undergo such micro-movements, i.e. micro-motion against each other in their contact areas. Corrosion and wear damage also occurs in the contact area between implant and bone through micro-movements and due to forces straining the bones.

During the treatment of the patient, corrosion and wear damages to the implanted osteosynthesis aids lead to the fact that metal ions from the implant components are released into the adjacent tissues. These ions may in part possess characteristics incompatible with the body and can thus lead to physical reactions such as, for example, inflammations, bone degeneration, healing disturbances and similar problems. Through these degradations of the implant, corrosion and wear damage can also occur, which lead for example to an increase in friction and sticking between two movable components of the implant. Corrosion and wear damage also leads to a decrease in the static and strength to a special degree in the dynamic strength.

### SUMMARY OF THE INVENTION

At the heart of the present invention is the object of providing an osteosynthesis aid used in trauma applications that decreases or avoids the occurrence of corrosion, abrasion and wear.

It is an additional object of the invention to increase the usefulness of an osteosynthesis aid or trauma device with the help of increased friction forces holding the device together.

The osteosynthesis aid has a section, which in its implanted state, is in contact with osseous tissue or metal. According to the invention, the surface, at least in this area, is modified in such a manner that corrosion and abrasion are decreased or avoided by a modification of the implant surface. The invention is based on the knowledge that corrosion and abrasion can be decreased or avoided. The invention is also based on the knowledge that a surface modification must not take place for the entire osteosynthesis aid, but that the surface modification in that section which is subjected in the implanted state to abrasion and corrosion is sufficient.

In a first preferred form of surface modification, the section of the osteosynthesis aid is modified by a coating. In one embodiment the coating osteosynthesis for all devices is applied through PVD (physical vapor deposition) or CVD (chemical vapor deposition). Here the DLC coatings (diamond-like carbon) and their subgroups are preferred, especially coatings of the metal, carbon and hydrogen type (MeC/CH), for example, coating with the components tantalum, carbon and hydrogen (TaC/CH). This coating is produced by a PVD and CVD process at a typical process temperature of 180° C.-200° C.

In another embodiment of the trauma device according to the invention, the modified section is hardened by a diffusion process. The diffusion process can be used in addition to the coating. Preferably through the diffusion procedure during a titanium alloying, nitrogen or oxygen for example (oxygen diffusion hardening) processes and in case of a steel alloying, such as 316 stainless steel, carbon for example, by kolsterizing are introduced into the section to be hardened. In kolsterizing 6-7 weight percent of carbon diffused into the metal surface in a chemo-thermic diffusion process at a temperature lower than 300° C. Such a process may be performed at Bodycote Kolsterising Apeldoorn Metal Technology (The Netherlands).

Another form of surface modification in the osteosynthesis aid provides for the use of an anodizing process. The anodizing process can lead especially to an anodization of Type II or of Type III through electrolytic treatment. Customarily, the electrolytic treatment is performed in special baths specifically provided for that purpose. Such a Type II anodization process is for example the “Dotize” process of the DOT Company (DOT Dunnschitht Und Obesflaechen-Technologies GmbH Rostock-Warnemunde Germany). The anodizing process can also be used in combination with other processes mentioned above, in order to obtain a sufficient surface modification.

In another embodiment of the osteosynthesis aid according to the invention, the section is modified by forming processes that either produce shavings or do not. Such treatment processes can contain the process steps of glass- and/or sandblasting. It is also possible to perform various blasting procedures with glass-, metal-, or ceramic spheres of different sizes with different blasting parameters. The processing procedure can also contain the working steps of slide-grinding. It is furthermore possible to smooth the surface to a special degree by mechanical polishing and/or electro-polishing.

In an especially preferred embodiment of the invention, an intramedullary nail is provided for an osteosynthesis aid, which in the implanted state, is affixed to the bone with the help of one or several bolting or cross-locking screws. According to the invention, the intramedullary nail and/or the bolting screw are provided with a surface modified as set forth above in their respective contact areas. The goal of this is to make sure that during rubbing between the intramedullary nail and the bolting screw, corrosion and wear of the two components do not occur.

In other preferred form, an intramedullary nail is provided, that, in the implanted state, accepts a femoral neck screw. According to the invention, the intramedullary nail and/or the femoral neck screw are provided with a surface modified as set forth above in their respective contact area.

In another preferred refinement of the osteosynthesis aid according to the invention, an intramedullary nail whose surface is modified in the contact area with the bone is intended as implant.

In another refinement, a hip plate is intended as osteosynthesis aid, that is held to the bone with a femoral neck screw and/or bolting screws, where the hip plate and/or the femoral neck screw as well as the bolting screw in their respective contact areas show a modified surface.

In addition to the abovementioned modified surface, the hip plate can have a modified surface in the area that is in contact with the osseous tissue.

The task according to the invention is also resolved through an osteosynthesis aid made from steel-, cobalt- and/or titanium alloy.

As an osteosynthesis aid, here an external fixation device with at a minimum a bone-pin, a construction rod and a clamping device for clamping the bone-pin and the rod is provided wherein the clamping area a modified surface is provided in such a way that the friction between clamping device and bone-pin as well as the bone pin, is held securely through the clamping device and that the risk of slipping is greatly reduced.

Advantageous design refinements of the osteosynthesis aid according to the invention are explained in more detail with reference to the figures in the following.

### BRIEF DESCRIPTION OF THE DRAWINGS

The invention can be performed in various ways and various embodiments will now be described by way of examples and with reference to the accompanying drawings in which:

FIG. 1 is an intramedullary nail with a femoral neck screw and bolting screws;

FIG. 2 is a schematic view of a hip plate with bolting screws and a femoral neck screw;

FIG. 3 is a schematic view of an intramedullary nail with a femoral neck screw; and

FIG. 4 is an external fixation with a bone-pin and a construction rod.

### DETAILED DESCRIPTION OF THE PREFERRED EMBODIMENTS

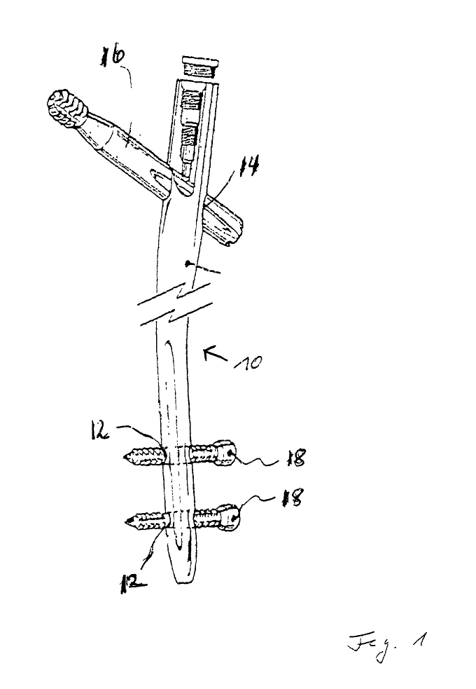

FIG. 1 shows a schematic view of an intramedullary nail 10. Such a nail is shown in U.S. Pat. No. 5,176,681. The intramedullary nail has two cross-locking through holes 12 at its distal end extending perpendicular to the longitudinal axis. At its proximal end, the intramedullary nail 10 has a through hole 14, running diagonally to the longitudinal axis. A femoral neck screw 16 is put into drill hole 14. The contact area between drill hole 14 and femoral neck screw 16 has a modified surface which leads to reduction of the damages based on movement of the femoral neck screw against the intramedullary nail. The surface modification is provided for on the femoral neck screw 16 as well as on the inner surface of drill hole 14, but this can also be done exclusively to the intramedullary nail or exclusively to the femoral neck screw. Cross-locking screws 18 extend through holes 12.

The drill hole 12 for the screws 18 is also equipped with a modified surface, in order to avoid wear and abrasion of material. Here too, the screws 18 can be provided with a modified surface.

For the surface modification, the advantageous effect of the invention can be obtained through a surface coating such as DLC coating as well as through surface hardening. Both surface modifications taken by themselves or in combination, ensure that, especially in case of lengthy dwell times of the intramedullary nail in the bone, no metal ions are released into the body and the nail used does not lose of any of its static and especially does not lose its dynamic strength.

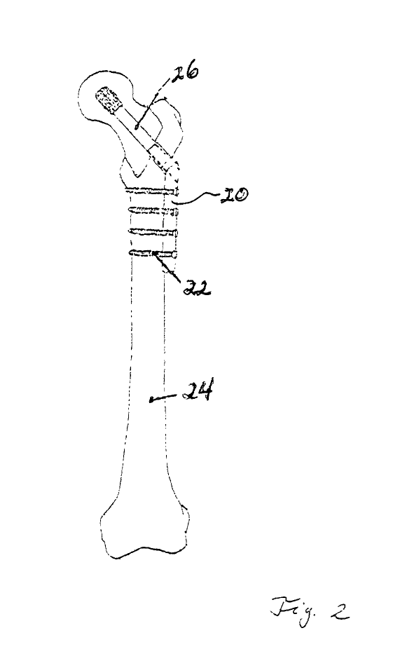

FIG. 2 shows a schematic view of a hip plate 20. The hip plate 20 has drill holes to accept bone screws 22. In the implanted state, the hip plate 20 lies against bone 24. In addition, the hip plate serves to tighten a femoral neck screw 26 which fixes a femoral neck fracture. Also when using hip plate 20, through movement and stressing of the bone 24, friction between the femoral neck screw 26, bone screw 22 and the bone material 24 occurs with respect to the hip plate 20. Also a surface contact exists from the femoral neck screw to the hip plate, where both can glide over each other, which leads to the occurrence of friction, wear and corrosion. Also screws 22 carry out micro-movements in the drill holes of hip plate 20, which lead to the occurrence of wear and corrosion. Added to this are the micro movements between the hip plate and the bone, which could possible lead to the release of metal ions. This can be effectively reduced or avoided through the surface modification of the hip plate.

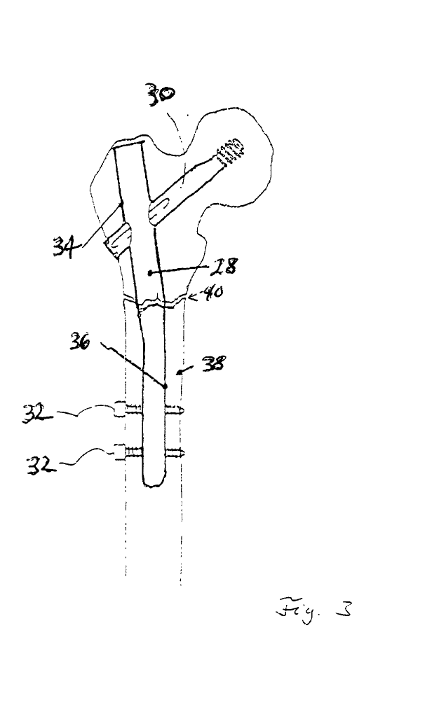

FIG. 3 shows a schematic view of an intramedullary nail 28 with a femoral neck screw 30 and two cross-locking screws 32. The intramedullary nail 34 is modified in the design example shown in FIG. 3 along its outer surface 36, which is in contact with osseous tissue 38. This is especially advantageous in case of a fracture 40, whose fractures line runs at the height of the shaft of the intramedullary nail. Through the modified surface 36, damaging the osseous tissue 38 can be effectively reduced or avoided.

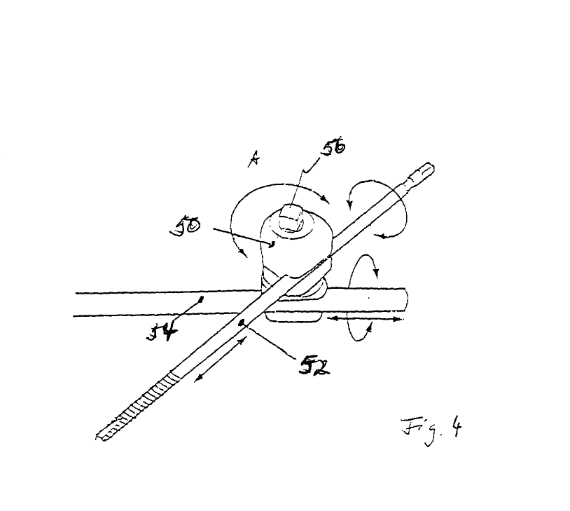

FIG. 4 shows a schematic view of an external fixator. Such a fixator is shown in U.S. Pat. No. 5,752,954. The external fixator has a clamp 50, with two openings to receive a bone pin 52 and a construction rod 54. The clamp is rotating along the direction marked by a double arrow A. Bone pin 52 and construction rod 53 are respectively arranged in clamp 50 and rotating in the lengthwise direction as well as around their longitudinal axis. By tightening the clamp by means of screw 56, the position of bone pin 53 and construction rod 54 is fixed. To obtain as great as possible a friction force between the elements and thus attain increased security against slipping out of construction rod 54 are provided with a modified surface in their contact areas. Clamp, bone pin and construction rod could also be provided by themselves with a modified surface. The modified surface increases the friction so that the clamping strength of clamp 50 is increased.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An osteosynthesis device made from a steel-, cobalt- and/or titanium alloy, comprising one section, which in its implanted state is in contact with osseous tissue or with metal has a surface which is modified in such a way that corrosion and abrasion are decreased or avoided.

2. The osteosynthesis device as set forth in claim 1, wherein the section is modified by means of a coating.

3. The osteosynthesis device as set forth in claim 2, characterized by the fact that the coating is applied by PVD (physical vapor deposition) or CVD (chemical vapor deposition).

4. The osteosynthesis device as set forth in claim 2, wherein coating is applied as DLC coating (diamond-like carbon).

5. The osteosynthesis device as set forth in claim 4, wherein the DLC layer is of the metal, carbon hydrogen type.

6. The osteosynthesis device as set forth in claim 1, wherein the section is hardened by a diffusion process.

7. The osteosynthesis device as set forth in claim 6, wherein the diffusion process introduces nitrogen, oxygen into the section in the case of a titanium alloy and carbon in the case of a steel alloy.

8. The osteosynthesis device as set forth in claim 1, wherein the section is modified through surface hardening and anodization.

9. The osteosynthesis device as set forth in claim 8, wherein the anodizing process produces an anodization of Type II or Type III through electrolytic treatment.

10. The osteosynthesis device as set forth in claim 1, wherein the section is modified through a shaping procedure.

11. The osteosynthesis device as set forth in claim 10, wherein the processing procedure contains the working step of glass- and/or sandblasting.

12. The osteosynthesis device as set forth in claim 10, wherein the processing step contains the working step of grinding, especially slide-grinding.

13. The osteosynthesis device as set forth in claim 11, wherein the processing procedure contains the working step of mechanical polishing and/or electro polishing.

14. The osteosynthesis device as set forth in claim 1, wherein the osteosynthesis aid comprises an intramedullary nail and at least one screw having a contact area which possess the modified surface.

15. The osteosynthesis device as set forth in claim 1, wherein the osteosynthesis aid comprises an intramedullary nail and a femoral neck screw having a contact area with said modified surface.

16. The osteosynthesis device as set forth in claim 1, wherein the osteosynthesis aid comprises an intramedullary nail having a modified surface in the contact area with the osseous tissue.

17. The osteosynthesis device as set forth in claim 1, wherein the device comprises a hip plate and a femoral neck screw each having said modified surface on their respective contact area.

18. The osteosynthesis device as set forth in claim 1, wherein said hip plate has at least one bone screw having said modified surface on a contact area.

19. The osteosynthesis device as set forth in claim 17, wherein said hip plate has a bone contacting surface with said aid.

20. The osteosynthesis device made from a steel- and/or titanium alloy, comprising an external fixator having a bone-pin, a rod and a clamping device wherein the clamping device, the bone-pin rod with modified surface contact such that the friction between clamping device and bone-pin rod is increased.

21. The osteosynthesis device as set forth in claim 5, wherein the DLC layer is of a tantalum, carbon and hydrogen type.

22. A static fracture fixation implant comprising:
a means for fixing the implant to bone;
a bone contacting surface, said surface having a coating selected from the group consisting of diamond-like carbon (DLC), diffusion hardening, anodization and combinations thereof.

23. The implant as set forth in claim 22, wherein the means for fixing the implant to bone is at least one bone screw.

24. The implant as set forth in claim 23, wherein the bone contacting surface includes a bone contacting surface on the bone screw.

25. The implant as set forth in claim 22, wherein the anodizing process produces an anodization of Type II or Type III through electrolytic treatment.

26. The implant as set forth in claim 22, wherein the DLC coating is of the metal, carbon, hydrogen type.

27. The implant as set forth in claim 26, wherein the metal is tantalum.

28. The implant as set forth in claim 22, wherein the implant is made of titanium or steel and wherein the diffusion hardening introduces nitrogen or oxygen into the bone contacting surface in the case of a titanium alloy and carbon in the case of a steel alloy.
